(19) United States
(12) Patent Application Publication
(10) Pub. No.: US 2020/0381780 A1
(43) Pub. Date: Dec. 3, 2020

(54) SOLID POLYMER ELECTROLYTE

(71) Applicant: DSM IP Assets B.V., Heerlen (NL)

(72) Inventors: Carel Frederik Constantijn FITIÉ, Echt (NL); Robert Hendrik Catharina JANSSEN, Echt (NL); Alexander Antonius Marie STROEKS, Echt (NL)

(21) Appl. No.: 16/970,512

(22) PCT Filed: Feb. 19, 2019
(86) PCT No.: PCT/EP2019/054129

§ 371 Date: Aug. 17, 2020

(30) Foreign Application Priority Data
Feb. 19, 2018 (EP) 18157488.0

(51) Int. Cl. H01M 10/0565; C07C 255/04; H01M 2/16; H01M 4/62

(52) U.S. Cl. CPC H01M 10/0565; C07C 255/04; H01M 2/1653; H01M 2300/0091; H01M 4/622; H01M 2300/0082; H01M 2/1646

(57) ABSTRACT

The invention relates to a polymer composition comprising: a) a thermoplastic copolyester comprising i. polyester hard segments in an amount of between 5 and 50 wt. %, with respect to the total weight of the polymer composition, and ii. soft segments having a number average molecular weight of between 2.000 and 10.000 g/mol; and b) a metal salt; and c) an organic nitrile component, and wherein the metal salt is present in a weight percentage between 10 to 80 wt. %, the organic nitrile component is present in a weight percentage between 10 and 80 wt. %, and the soft segment is present in a weight percentage between 10 and 80 wt. %, wherein the weight percentages are with respect to the total weight of metal salt, organic nitrile component and soft segment; as well as a battery comprising the polymer composition

This invention relates to a polymer composition useful as solid polymer electrolyte and a battery comprising the polymer composition.

Solid polymer electrolytes (SPE) are known and for example described in Qingwen Lu et al, Journal of Membrane Science 425-426 (2013) 105-112. This document describes a polysulfone (PSF) poly(ethylene oxide) (PEO) electrolyte and succinonitrile (SN) as solid solvent to dissolve lithium salts. A drawback of this system is that the conductivity is still insufficient and the amount of amorphous phase in the system is very high. This system still exhibits insufficient mechanical properties. Moreover, the polysulfone (PSF) poly(ethylene oxide) (PEO) electrolyte is difficult to prepare and PSF-based systems require high processing temperatures, which limits its potential to use in applications.

Alternative SPE films comprising succinonitrile are also known, and for example described in US2014/0255772. These systems are based on crosslinked polyethers. These systems are cumbersome to prepare as after mixing, crosslinking has to take place, which prohibits further processing into goods. Also, mechanical properties as disclosed in US2014/0255772 are insufficient, as elongation at break and tensile strength are insufficient.

Solid polymer electrolytes (SPE) based on various hard segments and PEO as soft segments are also known and for example described in WO2017005903. This document describes an SPE based on a thermoplastic elastomer containing hard blocks containing polyester, polyamide or diamide and ionically conductive soft blocks and a metal salt. These SPEs, however, have a drawback that their ionic conductivity is insufficient, especially at lower temperatures, such as room temperature. This limits its application potential, especially at high charge and/or discharge rates.

It is thus an object of the present invention to provide a polymer composition which may act as a solid polymer electrolyte exhibiting a high conductivity and less of these drawbacks.

This object has been achieved by a polymer composition comprising:
a) a thermoplastic copolyester comprising
   i. polyester hard segments in an amount of between 5 and 50 wt. %, with respect to the total weight of the polymer composition, and
   ii. soft segments having a number average molecular weight of between 2.000 and 10.000 g/mol; and
b) a metal salt; and
c) an organic nitrile component, and wherein
the metal salt is present in a weight percentage between 10 to 80 wt. %, the organic nitrile component is present in a weight percentage between 10 and 80 wt. %, and the soft segment is present in a weight percentage between 10 and 80 wt. %, wherein the weight percentages are with respect to the total weight of metal salt, organic nitrile component and soft segment.

In a preferred embodiment, the metal salt is present in a weight percentage between 20 to 80 wt. %, the organic nitrile component is present in a weight percentage between 10 and 70 wt. %, and the soft segment is present in a weight percentage between 10 and 70 wt. % wherein the weight percentages are with respect to the total weight of metal salt, organic nitrile component and soft segment.

### FIGURES

In FIGS. 1-5 ternary diagrams are shown that indicate the composition ranges of embodiments of the present invention (grey areas) as well as the compositions of Comparative Experiments A-D (datapoints labelled CE A-D) and Examples 1-18 (datapoints labelled Ex 1-18). Compositions are expressed in weight percentages with respect to the total weight of metal salt, organic nitrile component and soft segment in the composition.

FIG. 1

An embodiment of the invention where the metal salt is present in a weight percentage between 10.0 to 80.0 wt. %, the organic nitrile component is present in a weight percentage between 10.0 and 80.0 wt. %, and the soft segment is present in a weight percentage between 10.0 and 80.0 wt. %, wherein the weight percentages are with respect to the total weight of metal salt, organic nitrile component and soft segment.

FIG. 2

Preferred embodiment of the invention where the metal salt is present in a weight percentage between 20.0 to 80.0 wt. %, the organic nitrile component is present in a weight percentage between 10.0 and 70.0 wt. %, and the soft segment is present in a weight percentage between 10.0 and 70.0 wt. %, wherein the weight percentages are with respect to the total weight of metal salt, organic nitrile component and soft segment.

FIG. 3

Preferred embodiment of the invention where the metal salt is present in a weight percentage between 10.0 to 80.0 wt. %, the organic nitrile component is present in a weight percentage between 10.0 and 32.5 wt. %, and the soft segment is present in a weight percentage between 10.0 and 80.0 wt. %, wherein the weight percentages are with respect to the total weight of metal salt, organic nitrile component and soft segment.

FIG. 4

Preferred embodiment of the invention where the metal salt is present in a weight percentage between 10.0 to 45.0 wt. % the organic nitrile component is present in a weight percentage between 10.0 and 80.0 wt. %, and the soft segment is present in a weight percentage between 10.0 and 80.0 wt. %, wherein the weight percentages are with respect to the total weight of metal salt, organic nitrile component and soft segment.

FIG. 5

Preferred embodiment of the invention where the metal salt is present in a weight percentage between 10.0 to 45.0 wt. %, the organic nitrile component is present in a weight percentage between 10.0 and 32.5 wt. %, and the soft segment is present in a weight percentage between 22.5 and 80.0 wt. %, wherein the weight percentages are with respect to the total weight of metal salt, organic nitrile component and soft segment.

Thermoplastic copolvester

The polymer composition comprises a thermoplastic copolyester, comprising
i. polyester hard segments in an amount of between 5 and 50 wt. % with respect to the total weight of the polymer composition, and
ii. soft segments having a number average molecular weight of between 2.000 and 10.000 g/mol.

Thermoplastic copolyesters are known as such and are for example obtainable from DSM under the trade name Arnitel®, and from Dupont under the trade name Hytrel®. Preferably, the polyester hard segments are present in an amount of between 7 and 40 wt %, and most preferred in an amount of between 10 and 35 wt %, wherein the weight percentage is with respect to the total weight of the polymer composition.

The terms “hard segments” and “soft segments” are well-known in the field of thermoplastic copolyesters and refer to particular segments along the polymer chain of the thermoplastic copolyester. Hard segments generally contain one or multiple repeat units of a high-strength engineering polymer and are substantially crystalline over the use temperature range of the thermoplastics copolyester. The melting point of the hard segment is preferably higher than 100° C., more preferably higher than 150° C. and most preferred higher than 200° C. Soft segments generally contain one or multiple repeat units of a soft, low glass transition polymer that is substantially amorphous over the use temperature range of the thermoplastic copolyester. The glass transition temperature of the soft segment is preferably lower than 25° C., more preferably lower than 0° C., even more preferably lower than −25° C. and most preferably lower than −50° C.

Melting temperature and glass transition temperature are measured during the second heating run, according to ISO 11357-1/-3 with a heating and cooling rate of 10° C./minute under nitrogen atmosphere.

Each polymer chain of the thermoplastic copolyester generally contains multiple hard and soft segments.

### Soft Segment

The soft segment has a number average molecular weight of between 2.000 and 10.000 g/mol. The soft segment preferably comprises PEO or polycarbonate. The soft segment may optionally include further types of soft, low glass transition polymers.

The number average molecular weight of the soft segment is preferably at least 2.500 g/mol, more preferably at least 3.000 g/mol, even more preferably at least 3.500 g/mol. Preferably the number average molecular weight of the soft segment is smaller than 10.000 g/mol, more preferably smaller than 9.000 g/mol, most preferred smaller than 8.000 g/mol. The number average molecular weight of the starting material for the synthesis of the thermoplastic elastomer is measured by a hydroxyl end group titration according to DIN EN 13926 after which the number average molar mass is calculated from the outcome of this analysis. Once incorporated in the thermoplastic elastomer, the number average molecular weight of the soft segment can be assessed by NMR-methods as known in the art.

Preferably, the soft segment comprises PEO. It is possible that the soft segments comprising PEO originate from a poly(ethylene oxide)-terminated poly(propylene oxide)diol. It is however preferred that the soft segments originate from a polyethylene oxide diol. Most preferably the soft segments of the thermoplastic elastomer comprise at least 80 wt. % of the poly(ethylene oxide) segments, more preferably at least 90 wt. %, even more preferably at least 98 wt. % most preferred 100 wt. % in which the weight percentage is with respect to the total weight of the soft segments of the thermoplastic elastomer.

The soft segments preferably comprise PEO and may comprise small amounts of randomly copolymerized co-monomers to suppress the crystallization of the soft segment. Examples of suitable co-monomers include propylene oxide, glycidyl ethers, etc. It is also possible that the soft segments comprise a chain extender, preferably a di acid. The advantage of using a chain extender is that long soft segments are obtained while chain regularity and, thus, crystallization are suppressed to allow higher ionic conductivity.

In another embodiment, the soft segment comprises polycarbonate. Preferably, the polycarbonate is an aliphatic polycarbonate, more preferably, the polycarbonate is poly(hexamethylene carbonate), poly(tetramethylene carbonate), pol(propylene carbonate) or copolymers of these aliphatic polycarbonates. This has the advantage that that the polymer composition displays good conductivity and high electrochemical stability, enabling the use high voltage cathode materials in batteries comprising the polymer composition.

The weight percentage of the soft segment in the thermoplastic copolyester is preferably higher than 20 wt. % more preferably higher than 30 wt. %, still more preferably higher than 40 wt. %, most preferably higher than 50 wt. %, in which weight percentage is with respect to the total weight of the thermoplastic copolyester.

### Polyester Hard Segments

The polyester hard segments are present in an amount of between 10 and 50 wt. % with respect to the total weight of the polymer composition. The polyester hard segments are suitably built up from repeating units derived from at least one alkylene diol and at least one aromatic dicarboxylic acid or an ester thereof. The alkylene diol may be a linear or a cycloaliphatic alkylene diol. The linear or cycloaliphatic alkylene diol contains generally 2-6 C-atoms, preferably 2-4 C-atoms. Examples thereof include ethylene glycol, propylene diol and butylene diol. Preferably ethylene diol or butylene diol are used, more preferably 1,4-butylene diol. Examples of suitable aromatic dicarboxylic acids include terephthalic acid, 2,6-naphthalenedicarboxylic acid, 4,4′-biphenyldicarboxylic acid or combinations of these. The advantage thereof is that the resulting polyester hard segment is generally semi-crystalline with a melting point of for example above 120° C., preferably above 150° C., and more preferably of above 200° C. The polyester hard segments may optionally further contain a minor amount of units derived from other dicarboxylic acids, for example isophthalic acid, which generally lowers the melting point of the polyester. The amount of other dicarboxylic acids is preferably limited to not more than 10 mol %, more preferably not more than 5 mol %, in which mol % is with respect to the total number of moles of dicarboxylic acid monomer, so as to ensure that, among other things, the crystallization behaviour of the copolyesters is not adversely affected. The polyester hard segment is preferably built up from ethylene terephthalate, propylene terephthalate, and in particular from butylene terephthalate as repeating units. Repeating units built up from butylene terephthalate is also referred to as PBT. Advantages of these readily available units include favourable crystallisation behaviour and a high melting point, resulting in copolyesters with good processing properties, excellent thermal and chemical resistance and good puncture resistance.

### Metal Salt

The composition according to the invention contains one of the above described thermoplastic elastomers and a metal salt. The metal salt is a salt containing a cation of group la and Ila of the table of elements and as anion as for example ClO₄₋, SCN⁻, BF₄, As F₆⁻, CF₃SO₃₋, Br⁻, I⁻, PF₆⁻, (CF₃SO₂)₂N⁻, also known as TFSI, (CF₃SO₂)₃ C⁻, CF₃CO₂⁻, (FO₂S)₂N⁻, also known as FSI, bis(oxalate)borate, also known as BOB, as well as mixtures thereof. Preferred cations for the salts include Li⁺ for a lithium battery, and Na+for a sodium battery and Al³⁺ for Al batteries. Lithium, sodium, aluminium batteries, are batteries that have an anode comprising lithium, sodium respectively aluminium.

Preferably, the metal salt is Lithium bis(trifluoromethanesulfonyl)imide (LiTFSI), Lithium bis(fluorosulfonyl)imide (LiFSI), Lithium bis(oxalate)borate (LiBOB) and/or Lithium perchlorate, as these are readily soluble in the soft segment. Most preferred is LiTFSI, as this is readily available, chemically stable and very soluble in the soft segment.

### Organic Nitrile Component

The composition according to the invention contains an organic nitrile component. With “organic nitrile component” herein is understood an organic component comprising a nitrile functional group, also referred to as cyano functional group, such as for example acrylonitrile and propanenitrile. The organic nitrile component may be a component comprising multiple nitrile groups and/or be a mixture of more than one component comprising a nitrile group. Preferably, the organic nitrile component has a molecular weight lower than 2000 g/mol, more preferably lower than 1000 g/mol, even more preferably lower than 500 g/mol, and most preferred lower than 250 g/mol as this has the advantage that compositions with increased conductivity can be obtained. The molecular weight of the organic nitrile component can be determined by mass spectrometry method as known in the art. In a preferred embodiment of the invention, the organic nitrile component comprises an aliphatic dinitrile such as adiponitrile (AN) and/or succinonitrile (SN), as this has the advantage that the composition has increased thermal stability and shows high conductivity. Most preferred, the organic nitrile component is succinonitrile (SN) as this has the advantage that the composition displays increased conductivity in a wide temperature range.

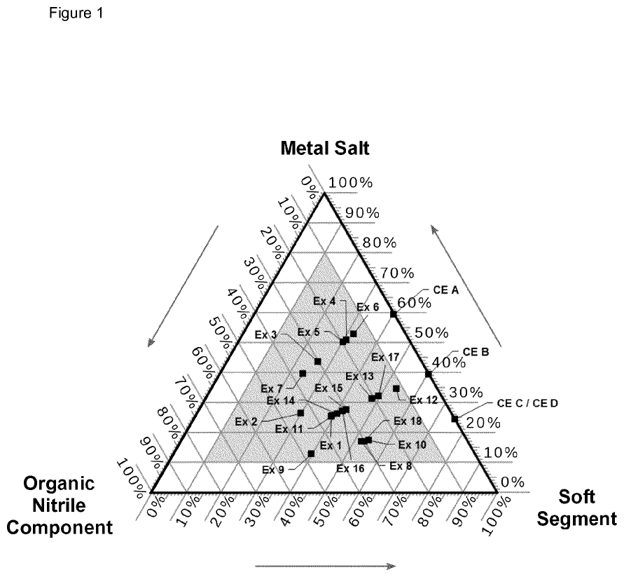

The inventors have found that with specific amounts of soft segment, metal salt and organic nitrile component, high conductivity can be reached, which is also shown in the examples. The metal salt is present in a weight percentage between 10 to 80 wt. %, the organic nitrile component between 10 and 80 wt. %, and the soft segment between 10 and 80 wt. %, wherein the weight percentage is with respect to the total weight of metal salt and organic nitrile component and soft segment (see FIG. 1). The total weight of the metal salt, organic nitrile component and soft segment add up to 100 wt. %. Solid polymer electrolytes comprising or even consisting of a polymer composition according to this embodiment have the advantage that very high ionic conductivity levels can be obtained at temperatures slightly above ambient conditions (50° C. and above). Such solid polymer electrolytes are especially well suited to make batteries that can operate under high (dis)charge rates.

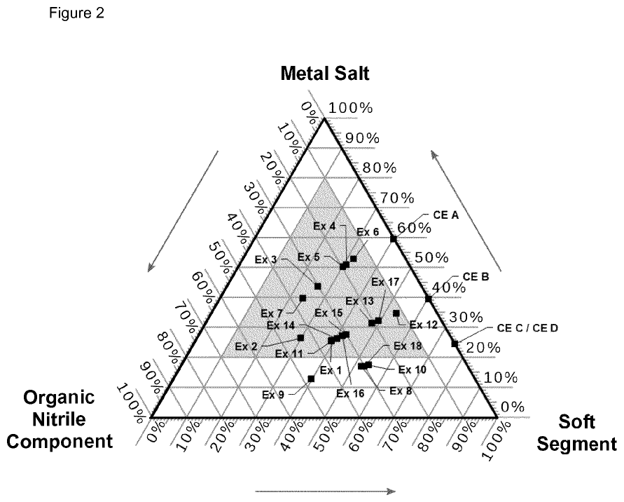

Preferably, the metal salt is present in a weight percentage between 20 to 80 wt. %, the organic nitrile component between 10 and 70 wt. %, and the soft segment between 10 and 70 wt. %, wherein the weight percentage is with respect to the total weight of metal salt and organic nitrile component and soft segment (see FIG. 2). Solid polymer electrolytes comprising or even consisting of a polymer composition according to this preferred embodiment have the advantage that they show less tendency to phase separate at temperatures below room temperature. Such solid polymer electrolytes have conductivity and mechanical performance that are more constant and robust when exposed to temperature changes, making them especially suitable for batteries with relatively temperature independent performance around ambient conditions.

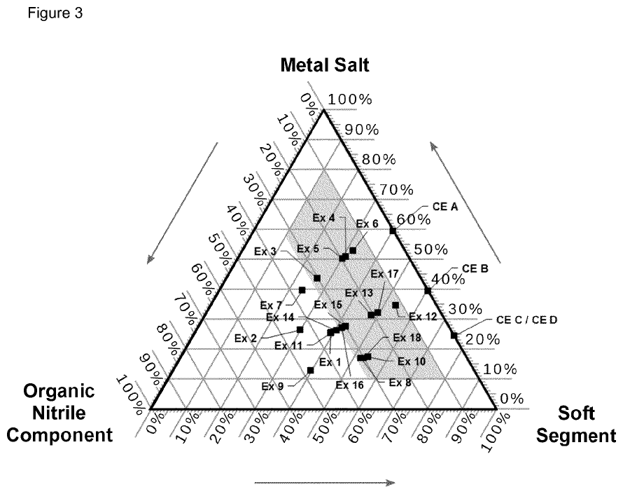

In another preferred embodiment of the invention the metal salt is present in a weight percentage between 10.0 to 80.0 wt. %, the organic nitrile component is present in a weight percentage between 10.0 and 32.5 wt. % and the soft segment is present in a weight percentage between 10.0 and 80.0 wt. % wherein the weight percentages are with respect to the total weight of metal salt, organic nitrile component and soft segment (see FIG. 3). Solid polymer electrolytes comprising or even consisting of a polymer composition according to this preferred embodiment have the advantage that very high ionic conductivity levels can be obtained at ambient conditions (around 20° C.). Such solid polymer electrolytes are especially well suited to make batteries that can operate under high (dis)charge rates at ambient conditions.

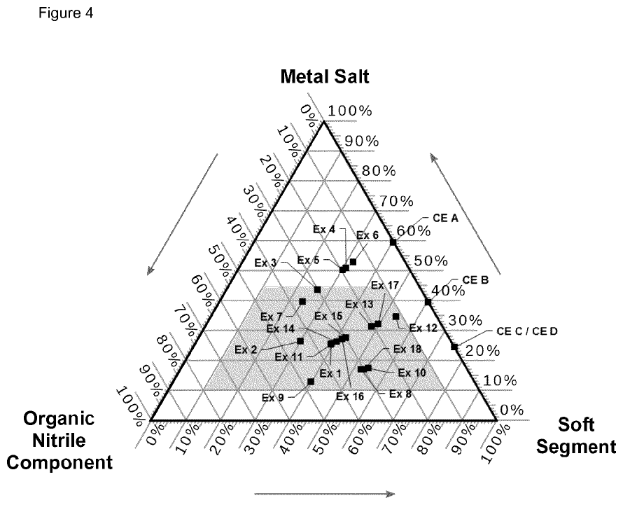

In yet another preferred embodiment of the invention the metal salt is present in a weight percentage between 10.0 to 45.0 wt. %, the organic nitrile component is present in a weight percentage between 10.0 and 80.0 wt. %, and the soft segment is present in a weight percentage between 10.0 and 80.0 wt. %, wherein the weight percentages are with respect to the total weight of metal salt, organic nitrile component and soft segment (see FIG. 4). Solid polymer electrolytes comprising or even consisting of a polymer composition according to this preferred embodiment have the advantage that acceptable conductivity levels and, thus, battery performance can be obtained with low amounts of metal salt. This allows to manufacture batteries in an economical way and with minimal environmental impact, since the most commonly used metal salts in battery applications are costly and contain substantial amounts of halogens.

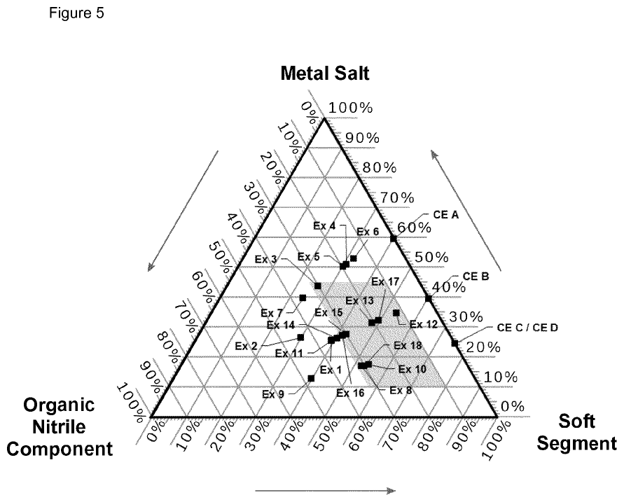

In yet another preferred embodiment of the invention the metal salt is present in a weight percentage between 10.0 to 45.0 wt. %, the organic nitrile component is present in a weight percentage between 10.0 and 32.5 wt. %, and the soft segment is present in a weight percentage between 22.5 and 80.0 wt. %, wherein the weight percentages are with respect to the total weight of metal salt, organic nitrile component and soft segment (see FIG. 5). Solid polymer electrolytes comprising or even consisting of a polymer composition according to this preferred embodiment have the advantage that acceptable conductivity levels and, thus, battery performance can be obtained at ambient conditions (around 20° C.) and with low amounts of metal salt. This allows to manufacture batteries for ambient temperature applications in an economical way and with minimal environmental impact, since the most commonly used metal salts in battery applications are costly and contain substantial amounts of halogens.

The invention also relates to a spacer between adjacent electrodes of a battery, especially of a rechargeable battery, the spacer comprising the polymer composition of the present invention. The polymer compositions of the present invention are especially advantageous, because they can be melt-processed in a film to act as spacer using standard polymer processing techniques as known in the art.

The invention also relates to an electrode, especially an electrode for a rechargeable battery, comprising the polymer composition of the present invention as a binder. Very good results are obtained when the polymer composition is used as a binder in the electrodes, especially in the cathode. This is because the binder with the polymer composition according to the invention is more conductive for ions, than known binders, so increasing the output of the battery, especially at low temperatures such as room temperature. In the electrode the binder acts to bind particles of active components, like for instance LiFePO₄ particles, preferably coated with carbon black, LiCoO₂ and Li(NiMnCo)O₂ particles. In case the particles are not coated with carbon black, preferably separate particles of a carbon-conductive agent, for instance carbon black or graphite, are incorporated into the cathode. The amount of binder used in porous electrodes may be between 2,5 and 20 wt. % and is preferably between 5 and 10 wt. % with respect to the total weight of the electrode.

The polymer compositions of the present invention are especially advantageous when used to make full solid-state batteries comprising non-porous electrodes and optionally Li-metal as anode. Such batteries have increased safety performance compared to batteries using volatile liquid electrolytes. In such an embodiment, the compositions of the present invention can be combined with active component particles and, optionally, other additives to form the electrode in a single melt-processing step. The amount of binder used in non-porous electrodes is 5-50 wt. % and is preferably 10-30 wt. % wherein wt % is with respect to the weight of the electrode, to produce cathodes that combine high capacity with good mechanical integrity.

The invention also relates to a battery, especially a rechargeable battery, comprising an adhesive film of the polymer composition according to the invention between the anode and/or the cathode at one hand and the spacer adjacent to at least one anode and/or at least one cathode at the other hand.

Very good results are obtained, even at room temperature, with a battery comprising an adhesive film of the polymer composition of the present invention between at least one anode and/or at least one cathode at one hand and the spacer adjacent to the at least one anode and/or at least one cathode at the other hand. This is because the contact resistance between the electrodes and the spacer is decreased. Especially good results are obtained with a ceramic spacer, the film filling the pores in the spacer.

### EXAMPLES

### Sample Preparation

Comparative Experiments A-C

Step 1: For CE B, 940 g. bis(trifluoromethanesulfonyl)imide lithium salt (LiTFSI) was dissolved in 458 g methanol (MeOH). This was added to 2062 g of thermoplastic copolyester (TPE) containing 70 wt. % PEO soft segment with a number average molecular weight of 4000 g/mol and 30 wt. % PBT hard segment into a 10L round bottom-flask. Using a rotary evaporator (a.k.a. rotavapor) set at a temperature of 60° C., the TPE granules and the liquid solution of LiTFSI in MeOH were tumbled together for 5 hours under nitrogen atmosphere until the granules visually looked dry and were free flowing. The MeOH was then removed at room temperature under reduced pressure and nitrogen gas purge in an oven. Final mass ratio based on the weights was 0.686 TPE and 0.314 LiTFSI.

Step 2: 24 g. of the granules made in step 1 were dosed to a pressing mold with dimensions 10×10×0.2 cm. A stack with a build-up as follows: lower pressing plate, Teflon sheet, pressing mold containing the granules, Teflon sheet and an upper pressing plate was placed into a press (Fontijne THB400). The press was closed and in 4 minutes heated to a temperature of 240° C. When temperature was reached the pressure is increased to 30 kN for a duration of 3 minutes. After these 3 minutes the press was opened, the stack taken out and placed in between two heavy metal objects in an oven under nitrogen atmosphere for cooling and to limit moisture uptake. After 10 minutes cooling the stack was dismantled and the solid polymer electrolyte plaque was cut out the pressing mold and sealed in an aluminum bag with a PE liner to prevent moisture uptake. The bag was purged with nitrogen gas just before.

This procedure was followed for all comparative experiments CE A-C with adjusted amounts of LiTFSI salt to obtain the compositions as shown in Table 1.

TABLE 1
Composition details sample preparation Comparative experiments
A-C

| | Composition | | | |
|---|---|---|---|---|
| | [wt % with respect to total polymer composition] | | | |
| | | Hard | | Organic Nitrile |
| Experiment | TPE | segment | Metal salt | component |
| CE A | 49.1% | 14.7% | 50.9% | 0.0% |
| CE B | 68.6% | 20.6% | 31.4% | 0.0% |
| CE C | 81.4% | 24.4% | 18.6% | 0.0% |

### Comparative Experiment D

Granules obtained in step 2 for CE C were processed to a film of approximately 50 μm thick and 30 cm wide using a laboratory scale film extrusion line operating at 220° C.

### Example 1

68.7 wt. % of thermoplastic copolyester (TPE) containing 70 wt. % PEO soft segment with a number average molecular weight of 4000 g/mol and 30 wt. % PBT hard segment was combined with 31,3 wt. % of LiTFSi into a film of 300-350 μm thickness via an extrusion process. The film was dried at 80° C. at reduced pressure with a small N₂ purge during the night and the weight was denoted. The dried film was submerged into liquid succinonitrile where the temperature of the succinonitrile was somewhere in between 70° C. and 80° C. for approximately 1 minute. After the 1 minute the film was taken out wiped clean with a dry cloth, briefly doped into acetone for removal of adjacent succinonitrile and dried again in an oven at 23° C. at reduced pressure with a small N₂ purge during the night after the weight was denoted again. Final mass ratio based on the measured weights was 0,480 TPE, 0,219 LiTFSi and 0,301 succinonitrile.

### Example 2

61.8 wt. % of thermoplastic copolyester (TPE) containing 70 wt. % PEO soft segment with a number average molecular weight of 4000 g/mol and 30 wt. % PBT hard segment was combined with 38.2 wt. % of LiTFSi into a film of 300-350 pm thickness via an extrusion process. The film was dried at 80° C. at reduced pressure with a small N₂ purge during the night and the weight was denoted. The dried film was submerged into liquid succinonitrile where the temperature of the succinonitrile was somewhere in between 70° C. and 80° C. for approximately 1 minute. After the 1 minute the film was taken out wiped clean with a dry cloth, briefly doped into acetone for removal of adjacent succinonitrile and dried again in an oven at 23° C. at reduced pressure with a small N₂ purge during the night after the weight was denoted again. Final mass ratio based on the measured weights was 0.381 TPE, 0.236 LiTFSi and 0.383 succinonitrile.

### Example 3-14

Granules and plaques of 5 cm×5 cm×350 μm were prepared following a procedure that was otherwise identical as described above for CE A-C. The plaques were submerged into liquid succinonitrile where the temperature of succinonitrile was somewhere in between 70° C. and 80° C. for 0.5-3 minutes in order to get samples differing in succinonitrile content. After this time the plaque was taken out wiped clean with a dry cloth, briefly doped into acetone for removal of succinonitrile on the surface of the plaque and dried again in an oven at 23° C. at reduced pressure with a small nitrogen purge for 2-3 hours after which the weight was denoted again. Samples with high (Ex 3-7), medium (Ex 11-14) and low (Ex 8-10) LiTFSI content were prepared by starting from plaques with compositions comparable to CE A, CE B, and CE C, respectively. Final mass ratios based on the measured weights of all samples were as shown in Table 2 below.

TABLE 2
Composition details sample preparation Examples 3-14

| | Composition | | | |
|---|---|---|---|---|
| | [wt % with respect to total polymer composition] | | | |
| | | Hard | | Organic Nitrile |
| Experiment | TPE | segment | Metal salt | component |
| Ex 3 | 33.9% | 10.2% | 39.3% | 26.8% |
| Ex 4 | 39.1% | 11.7% | 45.0% | 15.9% |
| Ex 5 | 38.4% | 11.5% | 44.5% | 17.1% |
| Ex 6 | 40.3% | 12.1% | 46.6% | 13.1% |
| Ex 7 | 31.2% | 9.4% | 36.2% | 32.6% |
| Ex 8 | 60.9% | 18.3% | 14.0% | 25.1% |
| Ex 9 | 48.8% | 14.6% | 11.1% | 40.1% |
| Ex 10 | 62.7% | 18.8% | 14.4% | 22.9% |
| Ex 11 | 48.2% | 14.4% | 22.0% | 29.9% |
| Ex 12 | 62.2% | 18.7% | 28.3% | 9.5% |
| Ex 13 | 57.0% | 17.1% | 26.0% | 17.0% |
| Ex 14 | 49.4% | 14.8% | 22.5% | 28.0% |

### Example 15

Step 1: Granules were prepared following a procedure that was identical as described above in step 1 for CE A-C. The final mass ratio based on the weights was 0.687 TPE and 0.313 LiTFSI.

Step 2: 28.9 g. succinonitrile (SN) was added to 86.4 g of the granules made in step 1 into a 500 ml round bottom-flask. Using a rotavapor set at a temperature of approximately 80° C. the granules and the liquid SN were tumbled together for about 4-6 hours under nitrogen atmosphere until the granules visually looked dry and were free flowing. Final mass ratio based on the weights was 0.515 TPE, 0.235 LiTFSI and 0.251 SN.

Step 3: 15 g of the granules made in step 2 was melt extruded using a small-scale twin-screw extruder (TSE, by Xplore) at a temperature of 200° C. The rotation speed of the TSE was set to 150 RPM. Approximately 1 minute after dosing the granules to the pre-heated TSE via the hopper, the melt was extruded via the die on a steel plate covered with a Teflon sheet and cooled down by putting another Teflon sheet covered steel plate on top of the extruded strand followed by manual pressing. After this, the sample was collected in an aluminum bag with a PE liner and sealed to prevent moisture uptake. The bag was purged with nitrogen gas just before. The composition after extrusion remains unchanged (0.515 TPE, 0.235 LiTFSI and 0.251 SN) which was confirmed by NMR spectroscopy.

Step 4: 24 g. of material extruded in step 3 was cut in small pieces and dosed to a pressing mold with dimensions 10×10×0.2 cm. A stack with a build-up as follows: lower pressing plate, Teflon sheet, pressing mold containing the granules, Teflon sheet and an upper pressing plate was placed into a press (Fontijne THB400). The press was closed and in 4 minutes heated to a temperature of 200° C. When temperature was reached the pressure was increased to 30 kN for a duration of 3 minutes. After these 3 minutes the press was opened, the stack taken out and placed in between two heavy metal objects in an oven under nitrogen atmosphere for cooling and to limit moisture uptake. After 10 minutes cooling the stack was dismantled and the solid polymer electrolyte plaque was cut out the pressing mold and sealed in an aluminum bag with a PE liner to prevent moisture uptake. The bag is purged with nitrogen gas just before.

### Example 16-17

Tensile bars with dimensions according to IS0527-1BA standard were punched out of a plaque prepared according to the procedure described for CE B. The tensile bar was dried at 80° C. at reduced pressure with a small nitrogen purge during the night and the weight was denoted. The dried tensile bar was submerged into liquid succinonitrile (SN) where the temperature of SN was somewhere in between 70° C. and 80° C. for approximately 20-25 minutes and 5-6 minutes for Ex 16 and Ex 17, respectively. After this time the tensile bar was taken out wiped clean with a dry cloth, briefly doped into acetone for removal of SN remaining on the surface and dried again in an oven at 23° C. at reduced pressure with a small nitrogen purge for 2-3 hours after which the weight was denoted again. Final mass ratios based on the measured weights wer 0.506 TPE, 0.231 LiTFSI and 0.263 SN for Ex 16 and 0.584 TPE, 0.266 LiTFSI and 0.150 SN for Ex 17.

### Example 18

The film prepared according to the procedure described for CE D was submerged in succinonitrile (SN) for 10-30 seconds following a procedure otherwise identical as described for the plaques of Ex 3-14 above. The final mass ratios based on the measured weight of the film sample was 0.616 TPE, 0.141 LiTFSI and 0.243 SN.

### Conductivity Measurement

For determining the conductivity, a Novocontrol dielectric spectrometer was used. The basic equipment contained the following parts; an Alpha-A analyzer incl. sample cell, a Quatro temperature controller including cryo-system with gas heater, Dewar vessel including heater and pressure sensor and Edwards vacuum pump including pipes and sensors and an instrument controller with software (Windeta). A standard geometry of two gold plated electrodes with diameter of 40 mm was used.

The SPE sample was prepared in an aluminum cup. On top of the sample an aluminum foil with a diameter of 40 mm was placed such that the SPE sample was sandwiched between Aluminium foils after the edges of the cup were cut away. Finally the Alu sandwiched SPE sample was placed between gold plated electrodes in the sample cell after which the complex impedance Z*=Z′+iZ″ was measured in the frequency range of 10mHz to 10 MHz at temperatures ranging from −20° C. to +100° C. in steps of 10° C. Finally, the real Z′ and the imaginary Z″ part of the impedances were plotted in a Nyquist plot; from which the ionic conductivity (Sigma) is determined as the lowest Z′ for which Z″ displays a local minimum, according to:

Sigma=1/Z′ * I/A, with 1=sample thickness and A=sample area.

### Tensile Measurement

For Ex 15, tensile bars with dimensions according to IS0527-1BA standard were punched out of a plaque prepared as described above. For Ex 16-17, the tensile bars prepared by submersion in liquid succinonitrile as described above were used directly.

Tensile measurements using the tensile bars were carried out on Zwick 1474 tensile machine using a 1 kN load cell, LightXtens with optical markers as extensometer, Pneumatic grips Zwick 8195.05 1kN, a grip to grip distance of 45 mm, a LO of manually placed markers between 11 and 15 mm and a pre-load of 0.1 N. E-modulus was measured at a tensile speed of 1 mm·min⁻. Test speed was 500 mm·min⁻¹. E-modulus (E_{mod}) was determined using regression between 0.3 and 0.8% strain. The elongation and stress at break of the sample are reported as EaB and SaB, respectively.

### Dendrite Growth Measurement

Symmetric cells of lithium metal-solid polymer electrolyte-lithium metal were constructed in a glove box environment based on films prepared as described above for CE D and Ex 18. Cells were allowed to rest for 5 hours. The lithium metal surface was preconditioned by applying five cycles of 1 hour stripping and plating steps at 0.05 mAcm⁻² followed by 1 hour rest at open circuit voltage (OCV) between each step. Dendrite growth measurements were conducted by applying a DC current of 0.1 mAcm⁻² and measuring the time until the first short circuit event was detected. All samples were measured in six-fold and short circuit times were reported as the average value±the standard deviation.

TABLE 3
Conductivity results

| | Comparative Data ^{*1} | Example 1 | Example 2 |
|---|---|---|---|
| T, ° C. | Sigma, S/cm | Sigma, S/cm | Sigma, S/cm |
| 70 | 8.5E−04 | 2.2E−03 | 3.1E−3 |
| 60 | 6.5E−04 | 1.6E−03 | 2.4E−3 |
| 50 | 3.8E−04 | 1.2E−03 | 1.7E−3 |
| 40 | 2.5E−04 | 8.1E−04 | 1.1E−3 |

^{*1} = Comparative data as taken from Qingwen Lu et al, Journal of Membrane Science 425-426 (2013) 105-112

Example 1 and 2 clearly show that with a solid polymer electrolyte consisting of the polymer composition according to the current invention superior conductivity levels are reached as compared to data reported in literature for a PSF-PEO system, especially at lower temperatures. Also, the mechanical properties of the solid polymer electrolyte remain sufficient.

TABLE 4
Composition overview and conductivity results

| | Composition | | | | | |
|---|---|---|---|---|---|---|
| | [wt % with respect to the total | | | | | |
| | weight of metal salt, organic | | | | | |
| | nitrile component and soft segment] | | | Conductivity | | |
| Examples/ | Metal | Organic nitrile | Soft | [Sigma, S/cm] | | |
| Experiments | salt | component | segment | 20° C. | 50° C. | 70° C. |
| CE A | 59.7% | 0.0% | 40.3% | 1.3E−06 | 3.3E−05 | 1.3E−04 |
| CE B | 39.5% | 0.0% | 60.5% | 7.4E−06 | 1.1E−04 | 3.3E−04 |
| CE C | 24.6% | 0.0% | 75.4% | 6.8E−06 | 1.1E−04 | 2.8E−04 |
| CE D | 24.6% | 0.0% | 75.4% | | | |
| Ex 1 | 25.6% | 35.2% | 39.3% | | 1.2E−03 | 2.2E−03 |
| Ex 2 | 26.6% | 43.2% | 30.1% | | 1.7E−03 | 3.1E−03 |
| Ex 3 | 43.8% | 29.8% | 26.4% | 1.5E−05 | 2.1E−03 | |
| Ex 4 | 51.0% | 18.0% | 31.0% | 2.4E−04 | 1.1E−03 | 2.2E−03 |
| Ex 5 | 50.3% | 19.4% | 30.4% | 1.4E−04 | 8.3E−04 | 1.8E−03 |
| Ex 6 | 53.1% | 14.9% | 32.1% | 3.3E−04 | 1.4E−03 | 2.8E−03 |
| Ex 7 | 39.9% | 36.0% | 24.1% | 1.1E−06 | 2.8E−03 | 2.8E−03 |
| Ex 8 | 17.1% | 30.7% | 52.2% | 5.4E−05 | 2.7E−04 | 5.6E−04 |
| Ex 9 | 13.0% | 47.0% | 40.0% | 3.9E−08 | 7.9E−04 | 1.4E−03 |
| Ex 10 | 17.7% | 28.2% | 54.1% | 3.9E−05 | 2.1E−04 | 4.4E−04 |
| Ex 11 | 25.7% | 34.9% | 39.4% | 3.2E−07 | 1.1E−03 | 2.0E−03 |
| Ex 12 | 34.8% | 11.7% | 53.5% | 1.7E−05 | 1.4E−04 | 3.6E−04 |
| Ex 13 | 31.4% | 20.5% | 48.2% | 3.3E−05 | 1.9E−04 | 4.5E−04 |
| Ex 14 | 26.4% | 32.9% | 40.6% | 7.9E−07 | 9.7E−04 | 1.9E−03 |
| Ex 15 | 27.8% | 29.7% | 42.6% | 7.8E−05 | 3.1E−04 | 6.9E−04 |
| Ex 16 | 27.2% | 31.0% | 41.8% | | | |
| Ex 17 | 32.3% | 18.2% | 49.6% | | | |
| Ex 18 | 17.3% | 29.8% | 52.9% | | | |

Table 4 provides an overview of the compositions of all the examples and comparative experiments, and the measured conductivity data at 20° C., 50° C. and 70° C. The results in Table 4 clearly show that all solid polymer electrolytes consisting of a polymer composition according to the current invention reach superior conductivity levels at temperatures of 50° C. and higher as compared to the comparative experiments CE A-C (see composition range in FIG. 1). Specifically, the solid polymer electrolyte of Ex 15 prepared via an extrusion process displays excellent conductivity, proving that solid polymer electrolytes consisting of a polymer composition according to the current invention are compatible with standard melt processing techniques.

The results in Table 4 further show that solid polymer electrolytes consisting of a polymer composition according to a preferred embodiment of the current invention show increased conductivity levels at room temperature (20° C., see composition range in FIG. 3). This is advantageous for applications in batteries where operation at ambient conditions is required.

The results in Table 4 also confirm that solid polymer electrolytes consisting of a polymer composition according to a preferred embodiment of the current invention show sufficient conductivity levels of >1.2 10⁻⁴ a S/cm at temperatures of 50° C. and above with a low salt content (see composition range in FIG. 4). This allows to manufacture batteries in an economical way and with minimal environmental impact, since the most commonly used metal salts in battery applications are costly and contain substantial amounts of halogens.

Lastly, the results in Table 4 also confirm that solid polymer electrolytes consisting of a polymer composition according to a further preferred embodiment of the current invention show acceptable conductivity levels of >1.5 10⁻⁵ S/cm with low salt content even at room temperature (20° C., see composition range in FIG. 5). This is advantageous to manufacture batteries that can operate under ambient conditions with the same benefits of economical production and minimal environmental impact

TABLE 5
Tensile test results

| | Composition | | | | | |
|---|---|---|---|---|---|---|
| | [wt % with respect to the total weight of metal | | | | | |
| | salt, organic nitrile component and soft segment] | | | Tensile properties | | |
| | Metal | Organic nitrile | Soft | E_{mod} | SaB | EaB |
| Example | salt | component | segment | [MPa] | [MPa] | [%] |
| Ex 15 | 27.8% | 29.7% | 42.6% | 6.7 | 4.3 | 803 |
| Ex 16 | 27.2% | 31.0% | 41.8% | 4.2 | 3.7 | 426 |
| Ex 17 | 32.3% | 18.2% | 49.6% | 6.2 | 5.2 | 727 |

The solid polymer electrolytes consisting of a polymer composition according to the current invention are all soft, rubbery materials that are highly suitable for battery applications. The tensile properties reported in Table 5 further confirm that the solid polymer electrolytes consisting of a polymer composition according to the current invention have excellent mechanical properties and, specifically, a very high elongation at break exceeding 400%. The advantage of such high elongation at break is that batteries with excellent mechanical integrity can be obtained.

TABLE 6
Dendrite growth results

| | Composition | | | |
|---|---|---|---|---|
| | [wt % with respect to the total weight of metal | | | |
| | salt, organic nitrile component and soft segment] | | | Short |
| | | Organic nitrile | | circuit time |
| Example | Metal salt | component | Soft segment | [hours] |
| CE D | 24.6% | 0.0% | 75.4% | 39 ± 14 |
| Ex 18 | 17.3% | 29.8% | 52.9% | 87 ± 12 |

The dendrite growth results reported in Table 6 show that the solid polymer electrolyte consisting of a polymer composition according to the current invention has a time to short circuit of about a factor two higher than the solid polymer electrolyte in comparative experiment CE D. This result demonstrates that solid polymer electrolytes consisting of a polymer composition according to the current invention have a superior resistance to the growth of lithium metal dendrites. This superior resistance is advantageous for solid polymer electrolyte applications in batteries that need high charge rates, especially when using metallic lithium as anode.

1. Polymer composition comprising:
a) a thermoplastic copolyester comprising
i. polyester hard segments in an amount of between 5 and 50 wt. %, with respect to the total weight of the polymer composition, and
ii. soft segments having a number average molecular weight of between

2. 000 and 10.000 g/mol; and
b) a metal salt; and
c) an organic nitrile component, and wherein the metal salt is present in a weight percentage between 10 to 80 wt. %, the organic nitrile component is present in a weight percentage between 10 and 80 wt. %, and the soft segment is present in a weight percentage between 10 and 80 wt. %, wherein the weight percentages are with respect to the total weight of metal salt, organic nitrile component and soft segment.

2. Polymer composition according to claim 1, wherein the metal salt is present in a weight percentage between 20 to 80 wt. %, the organic nitrile component is present in a weight percentage between 10 and 70 wt. %, and the soft segment is present in a weight percentage between 10 and 70 wt. %, wherein the weight percentages are with respect to the total weight of metal salt, organic nitrile component and soft segment.

3. Polymer composition according to claim 1, wherein the metal salt is present in a weight percentage between 10 to 45 wt. %, the organic nitrile component is present in a weight percentage between 10 and 32.5 wt. %, and the soft segment is present in a weight percentage between 22.5 and 80 wt. %, wherein the weight percentages are with respect to the total weight of metal salt, organic nitrile component and soft segment.

4. Polymer composition according to claim 1, wherein the soft segment comprises poly(ethylene oxide).

5. Polymer composition according to claim 1, wherein the soft segment comprises propylene oxide as co-monomer.

6. Polymer composition according to claim 1, wherein the soft segment comprises a PEO-PPO-PEO segment.

7. Polymer composition according to claim 1, wherein the soft segment comprises polycarbonate.

8. Polymer composition according to claim 1, wherein the molecular weight of the soft segment is between 3.000 and 8.000 g/mol.

9. Polymer composition according to claim 1, wherein the organic nitrile component is succinonitrile.

10. Polymer composition according to claim 1, wherein the polyester hard segment is PBT.

11. Polymer composition according to claim 1, wherein the metal salt is a Lithium bis(trifluoromethanesulfonyl)imide, Lithium bis(fluorosulfonyl)imide, Lithium bis(oxalate)borate or Lithium perchlorate, or any mixture thereof.

12. Spacer between adjacent electrodes of a battery, preferably of a rechargeable battery, the spacer comprising the polymer composition according to claim 1.

13. Electrode, preferably an electrode for a rechargeable battery, comprising the polymer composition according to claim 1.

14. Battery, preferably a rechargeable battery, comprising an adhesive film of the polymer composition according to claim 1 between an anode and/or a cathode at one hand and a spacer adjacent to the at least one anode and/or at least one cathode at the other hand.

15. Battery according to claim 14, wherein the spacer is a ceramic spacer.
